(19) United States
(12) Patent Application Publication
(10) Pub. No.: US 2026/0225099 A1
(43) Pub. Date: Aug. 6, 2026

(54) DEVICES AND METHODS FOR ISOLATING EXTRACELLULAR VESICLES

(71) Applicants: The Regents of the University of Michigan, Ann Arbor, MI (US); Northwestern University, Evanston, IL (US)

(72) Inventors: Scott Smith, Ann Arbor, MI (US); Sunitha Nagrath, Ann Arbor, MI (US); Harika Lingamarla, Ann Arbor, MI (US); Abha Kumari, Ann Arbor, MI (US); Adam Mendel Sonabend Worthalter, Chicago, IL (US); Mark Youngblood, Chicago, IL (US)

(21) Appl. No.: 19/530,966

(22) Filed: Feb. 5, 2026

Related U.S. Application Data
(60) Provisional application No. 63/754,225, filed on Feb. 5, 2025.

(51) Int. Cl. B01L 3/00; C12Q 1/6874; G01N 33/543; G01N 33/575; G01N 33/68

(52) U.S. Cl. CPC B01L 3/502761; B01L 3/502715; B01L 3/502753; C12Q 1/6874; G01N 33/543; G01N 33/5759; G01N 33/6872; B01L 2300/0893; B01L 2300/0896; C12Q 2600/106; G01N 2333/72; G01N 2570/00

(57) ABSTRACT

The present disclosure provides devices, systems, and methods for isolating extracellular vesicles utilizing extracellular vesicle capture agents. Particularly disclosed herein are devices, systems, and methods for isolating cancer-derived extracellular vesicles (e.g., meningioma (MGM)-specific extracellular vesicles).

### CROSS-REFERENCE TO RELATED APPLICATION

This application claims the benefit of U.S. Provisional Patent Application No. 63/754,225, filed Feb. 5, 2025, which is incorporated by reference herein in its entirety.

### FIELD

The present disclosure provides devices, systems, and methods for isolating extracellular vesicles utilizing extracellular vesicle capture agents. Particularly disclosed herein are devices, systems, and methods for isolating cancer-derived extracellular vesicles (e.g., meningioma (MGM)-specific extracellular vesicles).

### BACKGROUND

Extracellular vesicles are released by tumor cells and circulate in blood. Extracellular vesicles can aid in detection of malignancy following isolation. However, conventional methods of isolation are plagued by low throughput, insufficient isolation resulting in mixed extracellular vesicle population, poor yield of desired extracellular vesicles (e.g., disease-related extracellular vesicles), and damage to extracellular vesicles. Thus, disease-specific isolation methods that facilitate downstream analysis are needed.

### SUMMARY

Disclosed herein are microfluidic devices for isolating extracellular vesicles. In some embodiments, the microfluidic devices comprise: an inlet; an outlet; and a plurality of wells distributed between and fluidly connected to the inlet and outlet. In some embodiments, each of the plurality of wells in fluid communication with two or more adjacent wells downstream of the inlet by a connecting channel. In some embodiments, the plurality of wells, connecting channels, or a combination thereof are functionalized with an extracellular vesicle capture agent.

In some embodiments, the inlet is fluidly connected to a collection of inlet channels, wherein each inlet channel is directed to a different well proximal to the inlet.

In some embodiments, the plurality of wells is configured to uniformly distribute sample flow from the inlet channels to the plurality of wells.

In some embodiments, the plurality of wells is at least 2000 wells. In some embodiments, the plurality of wells is 4000 to 5000 wells.

In some embodiments, the plurality of wells has a volume capacity of greater than about 30 μL. In some embodiments, the plurality of wells has a volume capacity of about 30 μL to about 40 μL.

In some embodiments, each of the plurality of wells has a volume capacity of about 4 nL to about 6 nL.

In some embodiments, each of the plurality of wells has a diameter of about 200 μm to 400 μm.

In some embodiments, the microfluidic device has a capacity for up to about 100 μL of plasma. In some embodiments, the microfluidic device has a capacity for about 40 μL to about 100 μL of plasma.

In some embodiments, the extracellular vesicle capture agent is a binding partner of an extracellular vesicle marker, a cell marker, or a disease marker. In some embodiments, the extracellular vesicle capture agent is a binding partner of one or more membrane proteins. In some embodiments, the extracellular vesicle capture agent is an antibody, or fragment thereof.

Also disclosed herein are systems comprising a microfluidic device as disclosed herein and a fluid flow system.

Further disclosed herein are methods for isolating extracellular vesicles of interest. In some embodiments, the methods comprise: loading a sample comprising or suspected of comprising the extracellular vesicles of interest into a device or system as disclosed herein; and recovering the extracellular vesicles of interest, or components thereof, from the device.

In some embodiments, the sample is loaded at a flow rate of about 0.2 mL/hr to about 2.5 mL/hr. In some embodiments, the flow rate is about 1 ml/hr.

In some embodiments, the sample has a residence time in the device of about 30 seconds to about 5 minutes. In some embodiments, the sample has a residence time in the device of about 1 minute.

In some embodiments, the methods further comprise analyzing molecular signatures of the recovered extracellular vesicles of interest, or components thereof. In some embodiments, analyzing molecular signatures comprises proteomic analysis of proteins. In some embodiments, analyzing molecular signatures comprises nucleic acid sequencing. In some embodiments, the nucleic acid sequencing comprises RNA sequencing. In some embodiments, analyzing molecular signatures comprises detecting a disease biomarker.

In some embodiments, the sample is a biological sample. In some embodiments, the biological sample is obtained from a subject. In some embodiments, the subject has or is suspected of having a disease or disorder. In some embodiments, the methods further comprise diagnosing the disease or disorder. In some embodiments, the disease or disorder is cancer. In some embodiments, the methods further comprise assessing cancer status in the subject.

In some embodiments, the extracellular vesicles of interest are cancer-derived extracellular vesicles. In some embodiments, the capture agent is specific for the cancer-derived extracellular vesicles.

In some embodiments, the subject had or is having a medical procedure, treatment, or intervention. In some embodiments, the methods comprise comparing the molecular signature of the recovered extracellular vesicles of interest from the subject before and after the medical procedure, treatment, or intervention.

Additionally disclosed herein are methods for isolating meningioma (MGM)-specific extracellular vesicles. In some embodiments, the methods comprise contacting a biological sample comprising or suspected of comprising MGM-specific extracellular vesicles with somatostatin receptor 2 (SSTR2) antibodies; and recovering MGM-specific extracellular vesicles bound to the anti-SSTR2 antibodies. In some embodiments, the biological sample is obtained from a subject having or suspected of having meningioma. In some embodiments, the methods further comprise assessing meningioma status in the subject.

In some embodiments, the SSTR2 antibodies are immobilized on the solid support. In some embodiments, the solid support comprises a microfluidic device. In some embodiments, the SSTR2 antibodies are immobilized within wells of the microfluidic device.

In some embodiments, the methods further comprise analyzing molecular signatures of the recovered MGM-specific extracellular vesicles. In some embodiments, analyzing molecular signatures of the recovered MGM-specific extracellular vesicles comprises proteomic analysis of proteins. In some embodiments, analyzing molecular signatures of the recovered MGM-specific extracellular vesicles comprises nucleic acid sequencing. In some embodiments, the nucleic acid sequencing comprises RNA sequencing. In some embodiments, analyzing molecular signatures comprises detecting a disease biomarker.

In some embodiments, the subject had or is having a medical procedure, treatment, or intervention. In some embodiments, the methods comprise comparing the molecular signature of the recovered extracellular vesicles of interest from the subject before and after the medical procedure, treatment, or intervention.

Other aspects and embodiments of the disclosure will be apparent in light of the following detailed description and accompanying figures.

### BRIEF DESCRIPTION OF THE DRAWINGS

FIGS. 1A-1D show schematics and images of an exemplary ^{MGM}ExoChip microfluidic device. An ^{MGM}ExoChip drawn in AutoCAD is shown in FIG. 1A. A portion of the device was used to perform simulations in COMSOL (FIG. 1B). As shown, the top row has inlet flow at different locations. FIG. 1C is an SEM image of the device. The device has repeating patterns of 3 rows, as shown in pink, green, and purple colors. The microwells have a diameter of 300 μm. FIG. 1D is an image of a fabricated device.

FIGS. 2A and 2B show COMSOL simulations to calculate fluid velocity profiles (FIG. 2A) and trajectories (FIG. 2B). The fluid velocities are uniform across the device width and length, showing good distribution of flow (FIG. 2A). Flow velocity is lower in the wells to allow more time for interaction with the capture agent. Trajectories of 40 particles under drag and lift forces experienced by fluid flow are shown in different colors (FIG. 2B). The average residence time for this part of the chip was 4.5 sec. Each particle is distributed to a maximum of 3 branches.

FIG. 3 is a plot showing the effect of flow rate on the capture efficiency using ^{MGM}ExoChip.

FIG. 4 is a plot showing the effect of antibody concentration on the total protein quantity from extracellular vesicles using ^{MGM}ExoChip.

FIG. 5 is a plot of capture efficiency of vesicles from two meningioma cell lines, CH157 and IOMM, compared to a lung cancer cell line (A549) using ^{MGM}ExoChip with SSTR2 antibody.

FIG. 6 is an image of a western blot showing the specificity of the SSTR2 antibody for capturing meningioma specific extracellular vesicles. Two separate devices per condition and plasma from the same patient were used.

FIGS. 7A and 7B show SSTR2 specifically captures meningioma derived vesicles. FIG. 7A is a western blot showing that vesicles from patient plasma expressing SSTR2 were captured on an ^{MGM}ExoChip. No cellular contamination was observed. FIG. 7B is a plot showing that the protein amounts in vesicles isolated from high grade meningioma plasma (Patients 1 and 2) are higher than low grade (Patient 3). Healthy plasma was used as the control.

FIG. 8 shows the effect of plasma volume on the amount of protein isolated from meningioma vesicles using ^{MGM}ExoChip. The volume was diluted such that the total volume loaded onto the device was the same throughout.

FIG. 9A is a bar plot showing data collected in experiments testing capture efficiency using three different flow rates (0.6, 1.0, and 1.2 mL/hour) and three different cell lines (CH157, primary cells from grade 1 meningioma, and primary cells from grade 2 meningioma).

FIG. 9B is a bar plot showing data collected in experiments testing capture efficiency using SSTR2 and three other, different capture molecules: CD63, CD81, and CD9.

FIG. 9C is a photo taken with a light microscope of stained EV membranes after they were captured on ^{MGM}ExoChip.

FIG. 10A shows a western blot.

FIG. 10B shows a western blot.

FIG. 10C is a plot showing total protein amounts present in the grade 1 and grade 2 samples loaded onto the electrophoresis gels used for the western blots shown in FIG. 10A and FIG. 10B.

FIG. 10D is a plot and area under the receiver operating characteristic curve analysis.

FIG. 11A shows a volcano plot of differentially expressed genes.

FIG. 11B is a heatmap of differentially expressed genes presented after unsupervised clustering.

FIG. 11C is a cluster analysis plot.

FIG. 11D is a plot used for gene set enrichment analysis of differentially expressed genes.

FIG. 11E is a plot used for gene set enrichment analysis of differentially expressed genes.

FIG. 11F is a plot used for gene set enrichment analysis of differentially expressed genes.

### DETAILED DESCRIPTION

Disclosed herein are devices, systems, and methods for isolating cancer related or derived extracellular vesicles (e.g., exosomes) utilizing cancer-specific markers. Conventional ways of prognosing cancer and tumors include gene expression scores, DNA methylation, and DNA and RNA sequencing largely based on tissue biopsy. As tissue biopsy is invasive and not practical for all tumor locations, liquid biopsy provides a minimally invasive alternative. Extracellular vesicles (EVs) are nanometer sized vesicles and of particular interest because they carry cellular cargo and they are stable and abundant in plasma.

As described herein, meningioma (MGM)-specific EVs were isolated using an exemplary microfluidic device (^{MGM}ExoChip) exploiting antibodies to SSTR2, a highly expressed protein in meningioma cells. Using various characterization methods, a high capture efficiency of 80% was confirmed from model MGM cells-CH157 and two primary cells. A volume of 100 μL of a plasma sample was sufficient to extract an average of 7 μg of proteins and 5 ng of RNA without saturating the device. The extracellular vesicles were used for patients of high-grade and low-grade MGM and were able to show stratification of the MGM grades. Liquid biopsy and MGM extracellular vesicles are promising biomarkers for risk stratification of meningioma and determination of post-operative therapy.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of” the embodiments or elements presented herein, whether explicitly set forth or not.

For the recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The terms “antibody” and “antibodies” as used herein refer to monoclonal antibodies; monospecific antibodies (e.g., which can either be monoclonal, or may also be produced by other means than producing them from a common germ cell); multi-specific antibodies; human antibodies; humanized antibodies (fully or partially humanized); animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.); recombinant antibodies; chimeric antibodies; single-chain Fvs (“scFv”); single chain antibodies; single domain antibodies; Fab fragments; F(ab′) fragments; F(ab′)₂ fragments; disulfide-linked Fvs (“sdFv”); and anti-idiotypic (“anti-Id”) antibodies, dual-domain antibodies, dual variable domain (DVD), triple variable domain (TVD) antibodies (dual-variable domain immunoglobulins and methods for making them are described in Wu, C., et al., Nature Biotechnology, 25 (11): 1290-1297 (2007) and PCT International Application WO 2001/058956, the contents of each of which are herein incorporated by reference), or domain antibodies (dAbs) (e.g., such as described in Holt et al., Trends in Biotechnology 21:484-490 (2014)), and including single domain antibodies sdAbs that are naturally occurring, e.g., as in cartilaginous fishes and camelid, or which are synthetic, e.g., nanobodies, VHH, or other domain structure, and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity's sake, an antibody against an analyte is frequently referred to herein as being either an “anti-analyte antibody” or merely an “analyte antibody.”

“Antibody fragment” as used herein refers to a portion of an intact antibody that retains the ability to specifically bind to an antigen (see, generally, Holliger et al., Nat. Biotech., 23 (9): 1126-1129 (2005)) (e.g., comprises the antigen-binding site or variable region). Any antigen-binding fragment of the antibody described herein is within the scope of the present disclosure. The antibody may not include the constant heavy chain domains (e.g., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

Typically, an immunoglobulin or antibody is a protein that comprises at least one complementarity determining region (CDR). The CDRs form the “hypervariable region” of an antibody, which is responsible for antigen binding (discussed further below). A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2, and CH3) regions, and each light chain contains one N-terminal variable (VL) region and one C-terminal constant (CL) region. The light chains of antibodies can be assigned to one of two distinct types, either kappa (κ) or lambda (λ), based upon the amino acid sequences of their constant domains. In a typical antibody, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The VH and VL regions have the same general structure, with each region comprising four framework (FW or FR) regions. The term “framework region,” as used herein, refers to the relatively conserved amino acid sequences within the variable region that are located between the CDRs. There are four framework regions in each variable domain, which are designated FR1, FR2, FR3, and FR4. The framework regions form β sheets that provide the structural framework of the variable region (see, e.g., C. A. Janeway et al. (eds.), Immunobiology, 5th Ed., Garland Publishing, New York, N.Y. (2001)).

A “biomarker” or “marker” includes a biological compound, such as a protein and a fragment thereof, a peptide, a polypeptide, a proteoglycan, a glycoprotein, a lipoprotein, a carbohydrate, a lipid, a nucleic acid, that is present in a biological sample and that may be isolated from, or measured in, the biological sample. Furthermore, a biomarker may be the entire intact molecule, or a portion thereof that may be partially functional or recognized, for example, by an antibody or other specific binding protein. A biomarker may be associated with a given state of a subject, such as a particular stage of disease. In some embodiments, the biomarker is a cancer biomarker.

As defined herein, “a tumor” is a neoplasm that may either be malignant or non-malignant. Tumors of the same tissue type originate in the same tissue and may be divided into different subtypes based on their biological characteristics.

As used herein, the term “cancer” refers to a malignant disease caused or characterized by the proliferation of cells that have lost susceptibility to normal growth control. “Malignant disease” refers to a disease caused by cells that have gained the ability to invade either the tissue of origin or to travel to sites removed from the tissue of origin. Particular cancers related to the technology provided herein include, but are not limited to, lung cancer and pancreatic cancer.

A “subject” or “patient” may be human or non-human and may include, for example, animal strains or species used as “model systems” for research purposes, such as a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In some embodiments, the mammal is a human.

As used herein, the term “treating” includes reducing or alleviating at least one adverse effect or symptom of a disease or disorder through introducing in any way a therapeutic composition of the present technology into or onto the body of a subject. “Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods. For example, a “system” or “subsystem” may comprise one or more of, or any combination of, the following: mechanical devices, hardware, components of hardware, circuits, circuitry, logic design, logical components, software, software modules, components of software or software modules, software procedures, software instructions, software routines, software objects, software functions, software classes, software programs, files containing software, etc., to perform a function of the system or subsystem. Thus, the methods and/or steps of methods described herein, or certain aspects or portions thereof, may take the form of program code (e.g., instructions) embodied in tangible media, such as floppy diskettes, CD-ROMs, hard drives, flash memory, or any other machine-readable storage medium wherein, when the program code is loaded into and executed by a machine, such as a computer, the machine becomes an apparatus for practicing the embodiments. In the case of program code execution on programmable computers, the computing device generally includes a processor, a storage medium readable by the processor (e.g., volatile and non-volatile memory and/or storage elements), at least one input device, and at least one output device. One or more programs may implement or utilize the processes described in connection with the embodiments, e.g., through the use of an application programming interface (API), reusable controls, or the like. Such programs are preferably implemented in a high-level procedural or object-oriented programming language to communicate with a computer system. However, the program(s) can be implemented in assembly or machine language, if desired. In any case, the language may be a compiled or interpreted language and combined with hardware implementations.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. DEVICES AND SYSTEMS

The present disclosure provides microfluidic devices for use in isolating extracellular vesicles. The microfluidic devices may comprise an inlet, an outlet, and a plurality of wells distributed between the inlet and the outlet and fluidly connected to the inlet and the outlet.

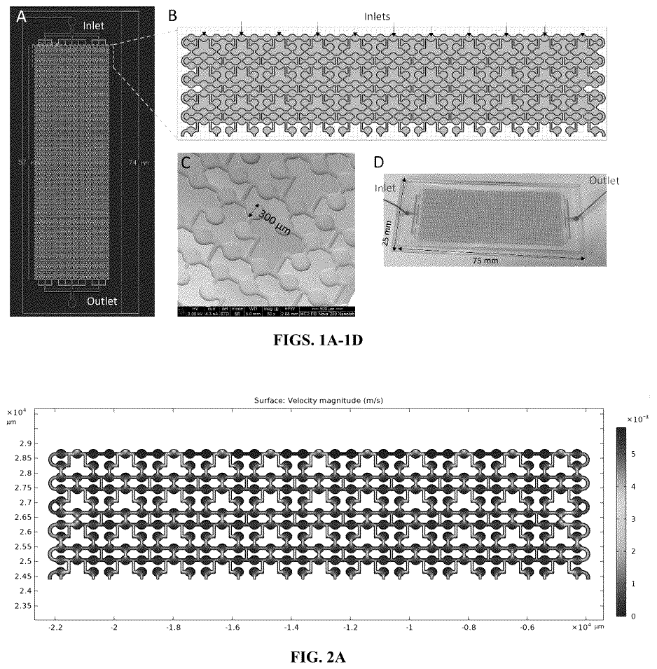

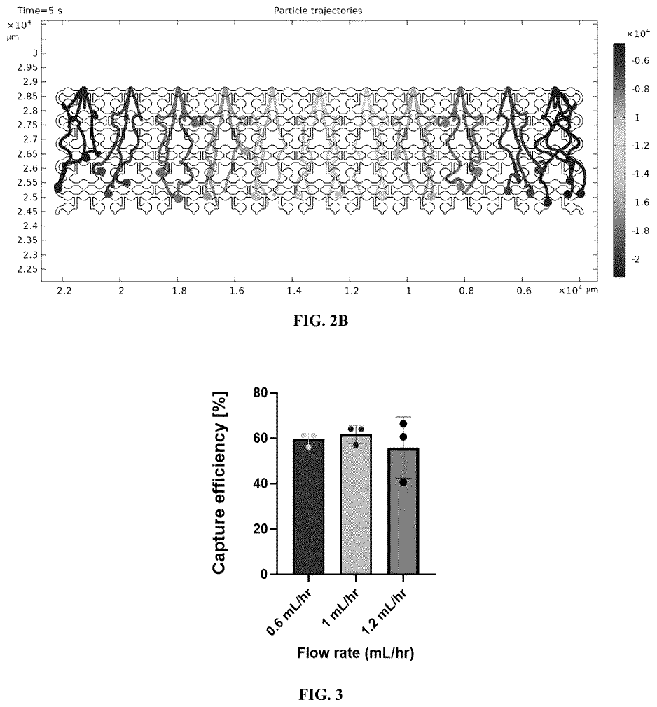

The wells are configured to uniformly distribute sample flow from the inlet channels to the plurality of wells. For example, the first wells fluidly connected to the inlet were not connected to wells immediately vertical or downstream in terms of fluid flow from inlet to outlet. Rather, the first wells fluidly connected to the inlet were fluidly connected to wells lateral to the fluid flow such that the fluid splits into multiple columns or branches instead of flowing directly below the inlet. This is shown in schematics for an exemplary device in FIGS. 1B and 2A. Other configurations may also be employed and can be confirmed for distribution of sample fluid flow using the methods disclosed herein in the Examples. Configurations of the wells can also affect residence time of the particles. Uniformly distributed sample flow facilitates a higher residence time of the sample in each of the wells and the overall device compared to a design with areas of low fluid distribution.

Any suitable number of wells may be included in a device. In some embodiments, the microfluidic device comprises at least about 2000 wells (e.g., greater than about 2000 wells, greater than about 2200 wells, greater than about 2400 wells, greater than about 2600 wells, greater than about 2000 wells, greater than about 2800 wells, greater than about 3000 wells, greater than about 3200 wells, greater than about 3400 wells, greater than about 3600 wells, greater than about 4000 wells, greater than about 4200 wells, greater than about 4400 wells, greater than about 4600 wells, greater than about 4800 wells, greater than about 5000 wells, greater than about 5200 wells, greater than about 5400 wells, greater than about 5600 wells, greater than about 5800 wells, greater than about 6000 wells, or more). In some embodiments, the microfluidic device comprises about 4000 to about 5000 wells (e.g., about 4100 wells, about 4200 wells, about 4300 wells, about 4400 wells, about 4500 wells, about 4600 wells, about 4700 wells, about 4800 wells, about 4900 wells, about 5000 wells).

The wells are arranged and configured such that fluid entering wells will have decreased but not zero flow. Thus, fluid entering the wells will slow down but continue flowing through and out of the well. The exact geometry of the wells may vary. In some embodiments, the wells are circular. In some embodiments, the wells have a flat top and/or a flat bottom. In some embodiments, the wells have a flat top and/or a flat bottom with rounded edges where they meet the walls of the wells. In some embodiments, the wells have a flat top and/or a flat bottom with squared edges where they meet the walls of the wells.

In some embodiments, each of the plurality of wells has a diameter of about 200 μm to 400 μm (e.g., about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, or about 400 μm).

In some embodiments, the plurality of wells has a volume capacity of greater than about 30 μL (e.g., about 35 μL, about 40 μL, about 45 μL, about 50 μL, about 55 μL, about 60 μL, about 65 μL, about 70 μL, or more) in total. In select embodiments, the plurality of wells has a volume capacity of about 30 μL to about 40 μL in total.

In some embodiments, each of the plurality of wells has a volume capacity of greater than about 1 nL (e.g., about 1 nL, about 2 nL, about 3 nL, about 4 nL, about 5 nL, about 6 nL, about 7 nL, about 8 nL, about 9 nL, or more) in total. In select embodiments, each of the plurality of wells has a volume capacity of about 4 nL, to about 6 nL in total.

The microfluidic device has a capacity for up to about 100 μL of plasma. For example, the microfluidic device may have a capacity for at least about 10 μL of plasma, at least about 20 μL of plasma, at least about 30 μL of plasma, at least about 40 μL of plasma, at least about 50 μL of plasma, at least about 60 μL of plasma, at least about 70 μL of plasma, at least about 80 μL of plasma, at least about 90 μL of plasma, but less than about 100 μL of plasma. In select embodiments, the microfluidic device has a capacity for about 40 μL to about 100 μL of plasma.

In some embodiments, each of the plurality of wells is in fluid communication with two or more adjacent wells downstream of the inlet by a connecting channel. A “channel,” as used herein, means a feature in the microfluidic device that directs the flow of a fluid (e.g., sample, wash fluid, and the like). The connecting channels can have any cross-sectional shape (circular, oval, triangular, irregular, square or rectangular, or the like). The connecting channel may be of any size. A connecting channel may also have any aspect ratio (length to average cross sectional dimension). The dimensions of the channel may be chosen such that fluid is able to freely flow through the microfluidic device from well to well. The dimensions and geometry of the channel may also be chosen, for example, to allow a certain volumetric or linear flowrate of fluid in the channel or to achieve a certain residence time within the channel. The geometry of the channels may vary depending on the location within the chip. The channels may be selected from: straight rectangular channels with two openings, channels with three openings e.g., that form a T shape, channels with two openings, e.g., that form an L shape, and channels with two openings at either end of a semicircle.

The connecting channel may include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the connecting channel may partially or completely fill the channel.

In some embodiments, the plurality of wells, connecting channels, or a combination thereof are functionalized with an extracellular vesicle capture agent or with a functionalization agent (e.g., a compound which allows tethering a capture agent of choice to the device). The capture agent can be directly bound to the surface of the wells and/or connecting channels using coupling agents such as bifunctional reagents or can be indirectly bound. Tethering of the capture to the surface of the plurality of wells and/or connecting channels may be accomplished by numerous methods known in the art, including, for example, avidin-streptavidin, biotin, and/or the use of a linker.

The extracellular vesicle capture agent may be any agent configured to specifically bind to extracellular vesicles of interest. In some embodiments, the capture agent is a binding partner (e.g., one half of a binding pair) that binds to an extracellular vesicle of interest, e.g., a binding partner (e.g., one half of a binding pair) that binds to a component of the extracellular vesicle. The binding partner has particular specificity for the target and under normal conditions binds to the target in preference to binding to other molecules. The interaction is typically non-covalent, but may also result in formation of a covalent bond. The binding partner may comprise a part of a larger molecule.

Exemplary binding partners include proteins, nucleic acids, glycoproteins, carbohydrates, hormones and the like. Specific examples of binding partner pairs (e.g., a first member of a binding pair/a second member of a binding pair) include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, etc. For instance, Protein A is a binding partner of the biological molecule IgG, and vice versa. Likewise, an antibody is a binding partner to its antigen, and vice versa. In some embodiments, the extracellular vesicle capture agent is or includes any moiety (e.g., nucleic acid, peptide, protein, or small molecule compound) that specifically binds to one or more membrane proteins.

In some embodiments, the extracellular vesicle capture agent is a binding partner of an extracellular vesicle marker, a cell marker, or a disease marker. Extracellular vesicle markers include those agents that are specific to extracellular vesicles or discriminate extracellular vesicles from other cellular components, such that extracellular vesicle capture agent binds to the extracellular vesicles preferentially over other cellular components. Extracellular vesicle markers may be those known in the art. See, e.g., Nonaka (2022) “Saliva Diagnostics” Annual Review of Analytical Chemistry 15:107-21; and van der Pol (2012) “Classification, functions, and clinical relevance of extracellular vesicles” Pharmacological Reviews 64:676-705, each of which is incorporated herein by reference. Exemplary extracellular vesicle markers may include CD9, CD63, CD81, ALIX, HSP70, TSG101, duramycin, heparin, and Annexin-V. Cell markers include those agents that may discriminate extracellular vesicles from one cell type versus another, such that extracellular vesicle capture agent binds to the extracellular vesicles from the desired cell type. Disease markers include those agents that may distinguish extracellular vesicles derived from one disease state from extracellular vesicles from another disease state or non-diseased state, such that extracellular vesicle capture agent binds to the extracellular vesicles from the desired disease state. In some embodiments, the extracellular vesicle capture agent binds a cancer marker. In some embodiments, the extracellular vesicle capture agent binds a meningioma marker. Exemplary cancer markers may include phosphatidylserine (PS), prostate-specific antigen (PSA), HER2, CA125, CEA, BRCA1/BRCA2, MCAM and/or MCSP, EGFR and SSTR2. In select embodiments, the extracellular vesicle capture agent is a binding partner of SSTR2, CD63, CD81, CD9, or Annexin-V.

In some embodiments, the extracellular vesicle capture agent is an antibody, or fragment thereof. The extracellular vesicle capture agent may be selected from a monoclonal antibody, a multispecific antibody, a bispecific antibody, a trispecific antibody, a tetravalent antibody, a single-domain antibody, a chimeric antibody, and a polyclonal antibody mixture. In some embodiments, the binding agent(s) are selected from a Fab, a scFv, a Fv, a scFv-Fc, a Fab′, a Fab′-SH, a F(ab′) 2, a diabody, a minibody, and a tribody. In select embodiments, the binding agent is a monoclonal antibody that specifically binds to an individual protein of interest. Preferably, the antibodies or fragments thereof are validated with high specificity and selectivity.

The wells, connecting channels, or a combination thereof may be functionalized with more than one capture agent. For example, multiple binding agents configured to bind to different binding partners of the same target extracellular vesicles may be used.

The microfluidic device may include one or more inlets for introducing one or more fluids (e.g., samples, wash fluids) into the devices. In some embodiments, the device comprises a single inlet. In some embodiments, the inlet is fluidly connected to a collection of inlet channels rather than directly to a single well of the microfluidic device. The collection of inlet channels allows the sample or other fluid to be directed to different wells proximal to the inlet. For example, each inlet channel is directed to a different well proximal to the inlet, such that the sample or fluid is distributed throughout a collection of wells proximal to the inlet. As described above for the connection channels, the inlet channels may have any cross-sectional shape, size, aspect ratio, etc. to allow for free flow of the sample or fluid into the section of the device comprising the plurality of wells.

The microfluidic device may include one or more outlets distal to the wells. In some embodiments, each of the wells proximal to the outlet may be connected to a single outlet.

The microfluidic device may be composed of any of a wide variety of materials, for example, polymers, plastics, resins, polysaccharides, silica or silica-based materials, carbon, metals, inorganic glasses, membranes, or any combinations thereof. The solid support material may be treated, coated, modified, printed or derivatized using polymers or chemicals to impart desired properties or functionalities to the support surface. Preferred solid support material may be compatible with the range of conditions encountered during the disclosed methods including salt concentrations, pHs, temperatures, and be optically transparent.

Devices disclosed herein may be fabricated using techniques known in the art. The techniques employed will depend on the material used to make the device. Exemplary techniques include, but are not limited to, molding, photolithography, electroforming, etching, and machining.

Also disclosed herein are systems comprising a microfluidic device as disclosed herein and a fluid flow system. For microfluidic assays, samples are often supplied by an operator using a micropipette. Fluid may be directed to flow through the device via one or more fluid flow systems. A fluid flow system can comprise compressors (e.g., providing positive pressure), pumps (e.g., providing negative pressure), actuators, and the like to control flow of the fluid. Fluid may also or otherwise be controlled using applied pressure differentials, centrifugal force, electrokinetic pumping, vacuum, capillary or gravity flow, or the like.

The fluid flow system may also facilitate pumping in of a sample or reagents (e.g., wash buffers, isolation buffers, and the like). For example, a sample or a first fluid is loaded into the microfluidic device, a second can be pumped in by disconnecting a line from the first pump and connecting a line from a second pump. Alternatively, valving may be used to switch from one pumped fluid to another. Different pumps may be useful to avoid cross contamination.

In some embodiments the systems comprise a receiving vessel or conduit for the fluid from the outlet. In some embodiments, the systems further comprise one or more sensors (e.g., pressure sensors, optical sensors, electrical sensors, etc.).

The fluid flow systems, receiving vessels, and/or sensors may be operatively coupled to one or more controllers that are, individually or collectively, configured to control and operate the system and/or microfluidic device. In some instances, the controller may be capable of receiving feedback (e.g., sensor data) from and/or controlling the fluid flow systems, receiving vessels, and/or sensors. The controller may be operatively coupled to a user interface configured to allow user input. The user interface may comprise an auditory or graphical user interface (e.g., display, screen, touchscreen, touchpad, lights, etc.). The user interface may comprise one or more user interactive devices, such as buttons, levers, knobs, keys, keyboards, mouse, joysticks, keypads, touchscreens, data ports, microphones, cameras, or other devices. The controller may be operatively coupled to output devices, such as a display, screen, printer, data port, speaker, light bulb, or the like.

### 3. METHODS

### Isolating Extracellular Vesicles

The present disclosure provides methods for isolating extracellular vesicles. The methods may comprise loading a sample comprising or suspected of comprising the extracellular vesicles of interest into a device as disclosed herein and recovering the extracellular vesicles of interest, or components thereof, from the device.

The device as disclosed herein is designed to operate at a higher flow rate than previously known devices. In some embodiments, the sample is loaded at a flow rate of about 0.2 mL/hr to about 2.5 mL/hr (e.g., about 0.5 ml/hr, about 1 ml/hr, about 1.5 ml/hr, about 2 ml/hr). In select embodiments, the flow rate is about 1 ml/hr.

The residence time, the time the sample is present in the device, is important for allowing sufficient time for the extracellular vesicles of interest and the capture agent to associate and form stable interactions. In some embodiments, the sample has a residence time in the device of about 30 seconds to about 5 minutes (e.g., about 30 seconds, about 1 minute, about 2 minutes, about 3 minutes, about 4 minutes or about 5 minutes). In select embodiments, the sample has a residence time in the device of about 1 minute.

In some embodiments, the methods further comprise washing bound extracellular vesicles with an appropriate buffer or solution that removes non-specific interactions with the capture agent after loading the sample and prior to recovering the extracellular vesicles of interest. Washing bound extracellular vesicles may be at flow rates higher than that used for loading the sample. Suitable washing buffers include those that do not damage the extracellular vesicles, e.g., PBS. In some embodiments, the washing buffers are stable in temperatures as low as −80° C.

In some embodiments, the methods further comprise applying an elution buffer or agent to release bound extracellular vesicles of interest, or components thereof, from the device. For example, the extracellular vesicles may be recovered intact or lysed for recovery of the components of the extracellular vesicles (e.g., with a suitable lysis buffer containing surfactants, detergents, and/or denaturants, such as RIPA buffer). For releasing intact extracellular vesicles, the extracellular vesicle capture agent may be functionalized or tethered to the device through a linker, e.g., a cleavable linker or a linker susceptible to being competed away from the extracellular vesicle capture agent.

The methods may further comprise analyzing or conducting a biological assay with the sample. For example, isolated extracellular vesicles of interest may be characterized using nanoparticle tracking analysis, scanning electron microscope (SEM), bicinchoninic acid (BCA) assay, western blot, and proteomics. In some embodiments, the methods may further comprise analyzing molecular signatures of the recovered extracellular vesicles of interest, or components thereof. In some embodiments, any of the methods include generating a report (such as a written or electronic report) disclosing a result of the molecular signature analysis (such as the presence, absence, or quantity of a molecular signature).

In some embodiments, the analysis further comprises isolating and analyzing the extracellular vesicles for the presence or amount of a marker (e.g., DNA or RNA or protein or any combination thereof) or antigen present. In some embodiments, assaying biomarkers comprises performing an immunoassay, e.g., to detect and/or quantify a biomarker. The technology is not limited in the type of immunoassay that is used to detect and/or quantify a biomarker and may include, e.g., an ELISA, a lateral flow immunoassay, a fluoroimmunoassay, a chemiluminescence immunoassay, or a western blot. In some embodiments, assays detect and/or quantify one or more of SSTR2, CD63, CD81, CD9, Flotillin-1, TSG101, and/or Annexin-V.

In some embodiments, the methods further comprise quantifying the extracellular vesicles in the sample.

The methods disclosed herein may be used for early diagnosis and screening of diseases and disorders or to aid in the diagnosis and screening of diseases and disorders, for example with other sample analysis. In some embodiments, the methods may further comprise diagnosing or prognosing a disease or disorder in a subject. In some embodiments, the disease or disorder is cancer.

The methods disclosed herein may be used for monitoring the progression of a disease or disorder. For example, the methods may be used for an initial assessment of disease status or grading. Multiple samples taken over time combined with analysis for molecular signatures of the recovered extracellular vesicles of interest, or components thereof, can be used to measure changes in the molecular signatures indicative of disease progression, regression, or maintenance of the disease status, state, or grade. In select embodiments, the methods may be used to assess and monitor cancer status.

The methods disclosed herein may also be used for monitoring the effect of a medical procedure, treatment, or intervention. In some embodiments, the methods may further comprise determining the success of the medical procedure, treatment, or intervention by comparing the molecular signature of the recovered extracellular vesicles of interest from the subject before and after the medical procedure, treatment, or intervention.

In some embodiments, the methods further comprise treating the subject based on the information obtained. For example, the subject may be administered one or more therapeutic agents (e.g., chemotherapeutic agents, corticosteroids, analgesics) or radiation or may undergo a surgery or other medical procedures. In some embodiments, the methods further comprise modifying treatment of the subject based on the information obtained. In some embodiments, the methods further comprise ceasing treatment of the subject based on the information obtained.

### Meningioma (MGM)-Specific Extracellular Vesicles

The present disclosure provides devices and methods for isolating meningioma (MGM)-specific extracellular vesicles. MGM is a tumor of the central nervous system, and MGMs comprise 39% of all primary intracranial tumors. MGM tumors are classified into three grades depending on their potency to become malignant. Risk stratification of MGM tumors plays a crucial role in determining post-operative therapy and treatment response. Conventional risk stratification uses gene expression scores, DNA methylation, and DNA and RNA sequencing and is based on tissue biopsy. As tissue biopsy is invasive and not practical for all tumor locations. The disclosed methods and devices are suitable for use with a liquid biopsy and provide minimally invasive alternatives.

The methods comprise contacting a biological sample comprising or suspected of comprising MGM-specific extracellular vesicles with a somatostatin receptor 2 (SSTR2) binding agent (e.g., a SSTR2 antibody) and recovering MGM-specific extracellular vesicles bound to the SSTR2 binding agent.

In some embodiments, the methods further comprise washing bound extracellular vesicles with an appropriate buffer or solution that removes non-specific interactions of extracellular vesicles after loading and prior to recovering. In some embodiments, the methods further comprise releasing bound extracellular vesicles of interest, or components thereof, from the device.

In some embodiments, the SSTR2 binding agent is immobilized on a solid support. “Solid support,” as used herein, refers any solid device or structure capable of immobilizing cells on a surface. For example, the solid support may be an array with spatially defined areas in which individual cells or cell types are isolated by a surface treatment or affinity methods (surface modifications or ligand binding). The solid support may have distinct structures (e.g., chambers, sections, wells, or channels) that separate the individual cells or cell types, for example, a microfluidic device or a microtiter plate.

The solid support may be composed of any of a wide variety of materials, for example, polymers, plastics, resins, polysaccharides, silica or silica-based materials, carbon, metals, inorganic glasses, membranes, or any combinations thereof. The solid support material may be treated, coated, modified, printed or derivatized using polymers or chemicals to impart desired properties or functionalities to the support surface. Preferred solid support material may be compatible with the range of conditions encountered during the disclosed methods including salt concentrations, pHs, temperatures, and be optically transparent.

The solid support may be smooth, having a substantially planar surface, or it may contain a variety of structures such as wells, grooves, depressions, channels, elevations, chambers, or the like, in which individual cells or cell types are isolated. The solid support may be a microfluidic device comprising a series of microchannels or chambers that isolate individual cells or cell types within the microchannels or within defined incubation chambers. The solid support may be a multi-well plate comprising a vast number of wells that isolate the individual cells or cell types. The solid support may be a particle or bead (e.g., a nanoparticle or microparticle). In some embodiments, the solid support is a microfluidic device. The disclosed microfluidic devices, and related methods, are suitable for use in the methods for isolating MGM-specific extracellular vesicles as described herein.

Similarly to described above, the methods disclosed herein may be used for early diagnosis and screening of meningioma or to aid in the diagnosis and screening of meningioma, for example with other sample analysis. In some embodiments, the methods may further comprise diagnosing or prognosing meningioma in a subject. The methods disclosed herein may be used for monitoring the progression of a meningioma. The methods disclosed herein may also be used for monitoring the effect of a medical procedure, treatment, or intervention for meningioma. In some embodiments, the methods further comprise treating the subject based on the information obtained.

### 4. Samples

As used herein, the term “sample” is used in its broadest sense. In one sense, it is meant to include a specimen obtained from any source, including biological and environmental samples. Biological samples may be obtained from animals (including humans) and encompass fluids, solids, and/or tissues. Such examples are not however to be construed as limiting the sample types. In some embodiments, the sample is a fluid sample such as a liquid sample. Examples of liquid samples suitable for use with the devices disclosed herein include bodily fluids (e.g., blood, serum, plasma, saliva, urine, ocular fluid, semen, sputum, sweat, tears, and spinal fluid), samples from supernatants and excreted materials of in vitro cultured tissues or cells, water samples (e.g., samples of water from oceans, seas, lakes, rivers, and the like), samples from home, municipal, or industrial water sources, runoff water, or sewage samples; and food samples (e.g., milk, beer, juice, or wine). Viscous liquid, semisolid, or solid specimens may be used to create liquid solutions, eluates, suspensions, or extracts that can be samples. Liquid samples can be made from solid, semisolid, or highly viscous materials, such as fecal matter, tissues, organs, biological fluids, or other samples that are not fluid in nature. For example, solid or semisolid samples can be mixed with an appropriate solution, such as a buffer, a diluent, and/or extraction buffer. The sample can be macerated, frozen and thawed, or otherwise extracted to form a fluid sample. Residual particulates may be removed or reduced using conventional methods, such as filtration or centrifugation. Samples can comprise biological materials, such as cells, microbes, organelles, and biochemical complexes. In some embodiments, the samples are cell-free, microbe-free, and/or organelle-free.

The biological sample may be obtained from any suitable subject, typically a mammal (e.g., dogs, cats, rabbits, mice, rats, goats, sheep, cows, pigs, horses, non-human primates, or humans). Preferably, the subject is a human. The sample may be obtained from any suitable biological source, such as a physiological fluid including, but not limited to, whole blood, serum, plasma, interstitial fluid, saliva, ocular lens fluid, cerebral spinal fluid, sweat, urine, milk, ascites fluid, mucous, synovial fluid, peritoneal fluid, vaginal fluid, menses, amniotic fluid, semen, feces, and the like. In some embodiments, the sample is blood or blood products. Blood products are any therapeutic substance prepared from human blood. This includes whole blood; blood components (e.g., red blood cell concentrates or suspensions; platelets produced from whole blood or via apheresis; plasma; serum and cryoprecipitate); and plasma derivatives (e.g., coagulation factor concentrates).

In some embodiments, the sample is a biological sample obtained from a subject having or suspected of having a disease or disorder. In some embodiments, the sample is a biological sample obtained from a subject having or suspected of having cancer. In some embodiments, the sample is obtained from a subject having or suspected of having meningioma.

In some embodiments, samples are obtained from a subject throughout the course of a disease or disorder or during treatment for a disease or disorder and the samples are analyzed for changes in the characterization of the extracellular vesicles over the time period of sample collection.

The term “cancer” refers to a class of diseases characterized by development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. The disclosed methods may be useful to wide range of cancer samples including carcinoma, sarcoma, lymphoma, leukemia, melanoma, mesothelioma, multiple myeloma, or seminoma. In select embodiments, the cancer may be meningioma. The cancer may be a cancer of the bladder, blood, bone, brain, breast, cervix, colon/rectum, endometrium, head and neck, kidney, liver, lung, lymph nodes, muscle tissue, ovary, pancreas, prostate, skin, spleen, stomach, testicle, thyroid, or uterus.

In some embodiments, the sample is a biological sample obtained from a subject who had or is having a medical procedure, treatment, or intervention. In some embodiments, a sample obtained from a subject before and after a medical procedure, treatment, or intervention and both are analyzed for changes in the characterization of the extracellular vesicles. Thus, the methods herein may be used to follow the success of the medical procedure, treatment, or intervention. The type of medical procedure, treatment, or intervention is not limited to the methods described herein. Exemplary medical procedures, treatments or interventions include: surgeries and surgical excisions or resections, transplants, biopsies, full or focal ablations (e.g., of a tumor or a tissue), administration of an active agent (e.g., pharmaceutical or therapeutic agent), insertion of a fiducial, brachytherapy seed, or drug depot, placement of devices (e.g., stents, grafts, implants, prosthetics), radiation therapy, and the like.

The sample may be used directly as obtained from the biological source or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, and the like. In some embodiments, the sample is enriched for extracellular vesicles.

### 5. Extracellular Vesicles

A unified vesicle nomenclature and classification system utilizing broadly accepted definitions has been elusive in the field. The term “extracellular vesicles,” as used herein, refers to a lipid membrane particles having a diameter (or largest dimension where the particles is not spheroid) of between about 30 nm and 10,000 nm. Extracellular vesicles encompass exosomes, ectosomes, microvesicles, microparticles, prostasomes, tolerosomes (which induce immunological tolerance to dietary antigens), apoptotic bodies (released by apoptotic cells), and nanovesicles. The term “exosome,” as used herein, refers to a membranous particle having a diameter between about 30 nm and 200 nm, wherein at least part of the membrane of the exosomes is directly obtained or derived from a cell. Most commonly, exosomes will have a size (average diameter) that is up to 5% of the size of the donor cell. Therefore, especially contemplated exosomes include those that are shed from a cell. As used herein, it is not intended that an extracellular vesicle or exosome of the invention be limited by any particular size or size range.

In some embodiments, the extracellular vesicles comprise exosomes. Exosomes may include any shed membrane bound particle that is derived from either the plasma membrane or an internal membrane. Exosomes can also include cell-derived structures bounded by a lipid bilayer membrane arising from both herniated evagination separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the exosome lumen including tumor-derived microRNAs or intracellular proteins. Exosomes can also include membrane fragments.

Extracellular vesicles may be released by mammalian cells for a number of purposes. During pregnancy, for example, exosomes inhibit the production of certain T-cells thereby protecting the fetus. In the case of certain bacterial infections, exosomes derived from infected cells express antigenic fragments of the bacterium to stimulate the immune system against the pathogen. It has been postulated that cancers use the immunomodulatory properties of exosomes in order to evade the immune system. The extracellular vesicles may be specific for a disease, disorder, or condition.

The type of originating cell can be used to describe the extracellular vesicles, e.g., the term “tumor-derived extracellular vesicles” or “tumor extracellular vesicles” or “cancer extracellular vesicles” refers to extracellular vesicles secreted by, derived from, and indicative of tumor, cancer, and/or malignant cells. Similarly, the term “normal extracellular vesicles” refers to exosomes secreted by, derived from, and indicative of normal cells. As used herein, “normal cells” refers to substantially healthy, non-diseased, non-apoptotic, and non-stressed cells (e.g., non-tumorigenic cells). The disclosed devices, systems and methods are suitable for use with any extracellular vesicles, e.g., secreted or from diseased (e.g., cancerous) cells or normal cells.

In some embodiments, the extracellular vesicles are cancer cell-derived extracellular vesicles. The cancer cells may be from any cancer, including, but not limited to, breast cancer, lung cancer, head & neck cancer, prostate cancer, esophageal cancer, tracheal cancer, brain cancer, liver cancer, bladder cancer, stomach cancer, pancreatic cancer, ovarian cancer, uterine cancer, cervical cancer, testicular cancer, colon cancer, rectal cancer, or skin cancer. In some embodiments, the cancer cells are meningioma cells.

### 6. Kits

Also within the scope of the present disclosure are kits that include the disclosed devices or one or more components necessary for making or using the disclosed devices, or for carrying out the disclosed methods. For example, in some embodiments, the kits include any or all of: non-functionalized microfluidic devices or sub-parts thereof and one or more extracellular vesicle capture agents, a disclosed device, a fluid flow system, buffers, and the like. The kits may further comprise a sample comprising extracellular vesicles.

In some embodiments, the kits may further contain materials for procuring or processing the sample.

Individual member components of the kits may be physically packaged together or separately. The components of the kits may be provided in bulk packages (e.g., multi-use packages) or single-use packages. The kits provided herein are in suitable packaging. Suitable packaging includes, but is not limited to, vials, bottles, jars, flexible packaging, and the like.

The kits can also comprise instructions for using the components of the kit. The instructions are relevant materials or methodologies pertaining to the kits. The materials may include any combination of the following: background information, list of components and their availability information (purchase information, etc.), brief or detailed protocols for using the compositions, troubleshooting, references, technical support, and any other related documents. Instructions can be supplied with the kits or as a separate member component, either as a paper form or an electronic form which may be supplied on computer readable memory device or downloaded from an internet website, or as recorded presentation.

It is understood that the disclosed systems or kits can be employed in connection with the disclosed methods.

### 7. EXAMPLES

### Materials and Methods

Flow Rate Analysis 2.4E8 per device extracellular vesicles were loaded each of three devices. The quantity of extracellular vesicles recovered from each outlet were subtracted from corresponding inlet value to estimate of capture efficiency. Washing conditions were 1 mL/hr (for 0.6 mL/hr), 1.5 mL/hr (for 1 mL/hr), and 1.8 mL/hr (for 1.2 mL/hr) with PBS as the buffer (values in parentheses are the X axis labels FIG. 3 and correspond to capture flow rates).

Antibody Concentration Analysis 300 uL of diluted plasma (100 uL plasma+200 uL PBS) was loaded in each of three devices for the indicated antibody concentrations. Following washing, protein extraction buffer (RIPA) was used to recover components from the bound extracellular vesicles in the outlet. The recover proteins were quantified. Capture flow rate was 1 mL/hr and wash flow rate was 1.5 mL/hr.

Cell Line Analysis 2.4E8 per device extracellular vesicles from each of the indicated cell lines were loaded each of two devices. Flow conditions were 1 mL/hr loading and 1.5 mL/hr wash. Capture efficiency was determined as in the flow rate analysis.

Plasma Experiments 300 uL of diluted plasma sample, with a standard dilution of 100 uL plasma to 200 uL PBS unless otherwise indicated, was loaded at 1 mL/hr and 1.5 mL/hr wash conditions. Extracellular vesicle proteins were recovered with RIPA buffer and quantified. A fixed amount of protein was loaded for western blot.

### Example 1

### ^{MGM}ExoChip

FIG. 1 shows an exemplary microfluidic device design in AutoCAD, the model used for COMSOL simulations, and SEM image. ^{MGM}ExoChip is a microfluidic platform with a polydimethylsiloxane top and a glass bottom, functionalized with neutravidin-biotinylated SSTR2. The exemplary device is 74 mm long and 24 mm wide with the specifications shown in Table 1 below, in comparison to a related design from Kang Y T, et al., Small. 2019 November;15 (47): e1903600.

^{MGM}ExoChip has 4224 wells compared to the device of Kang. The device disclosed herein has a higher volume capacity, 40% more than Kang. There are extra connectors (2900 in number) that make fluid flow more spread out and increase residence time by 7 times. Wells occupy 3.8 times more area than connectors. The new design has a higher throughput as it is able to operate at a higher flow rate.

TABLE 1

| Specifications | Kang et al. | | ^{MGM}ExoChip | |
|---|---|---|---|---|
| Number of wells | 1800 | | 4224 | |
| Well diameter | 500 | um | 300 | um |
| Volume capacity | 25 | uL | 35 | uL |
| Optimal flowrate operation | 0.6 | mL/hr | 1 | mL/hr |
| Residence time | 8 | sec | 59 | sec |

| | | |
|---|---|---|
| Design type | Straight flow | Distributed flow |
| | (along one line) | (splits into a maximum |
| | | of 3 branches) |

COMSOL was used to simulate fluid flow profile and study particle trajectory under that fluid flow. FIG. 2A shows that the fluid is uniformly distributed from the inlet. The velocity magnitude is in the order of mm/s which is typical in microfluidic systems. The pressure drop in this section of the device is 14.06 Pa. Hence, pressure drop along the channel length is about 150 Pa.

The typical size of extracellular vesicles (EVs) is a few nm to 200 nm (150 nm was used for calculations). FIG. 2B shows that particles of size 200 nm experience drag and lift forces under fluid flow and have an average residence time of 4.5 sec. Hence, the total residence time of these particles at 1 mL/hr flow rate is 59 sec or about a min. This value is 4.5 times higher than the residence time of the same particle in a rectangular channel.

### Example 2

### Capture Efficiency

Extracellular vesicles were isolated from an MGM cell-line (CH157), primary cell-derived EVs, and patient plasma and characterized using nanoparticle tracking analysis, SEM, BCA, western blot, and proteomics.

RNA extraction was performed using Norgen Biotek kit, sequenced in Illumina platform at 40M raw reads, and data was analyzed using R. Statistical analyses were performed using Student's t-test.

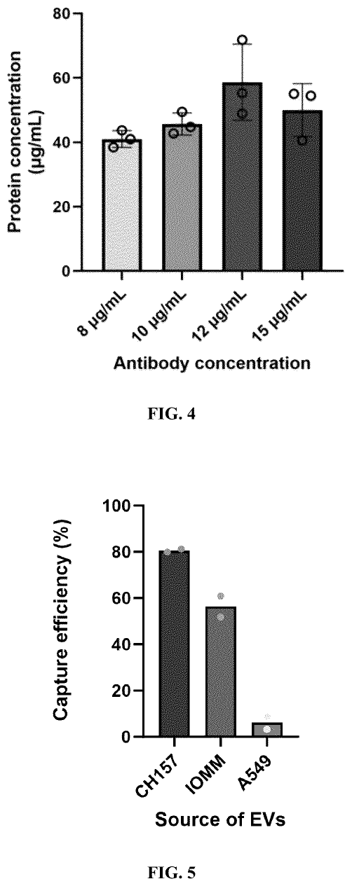

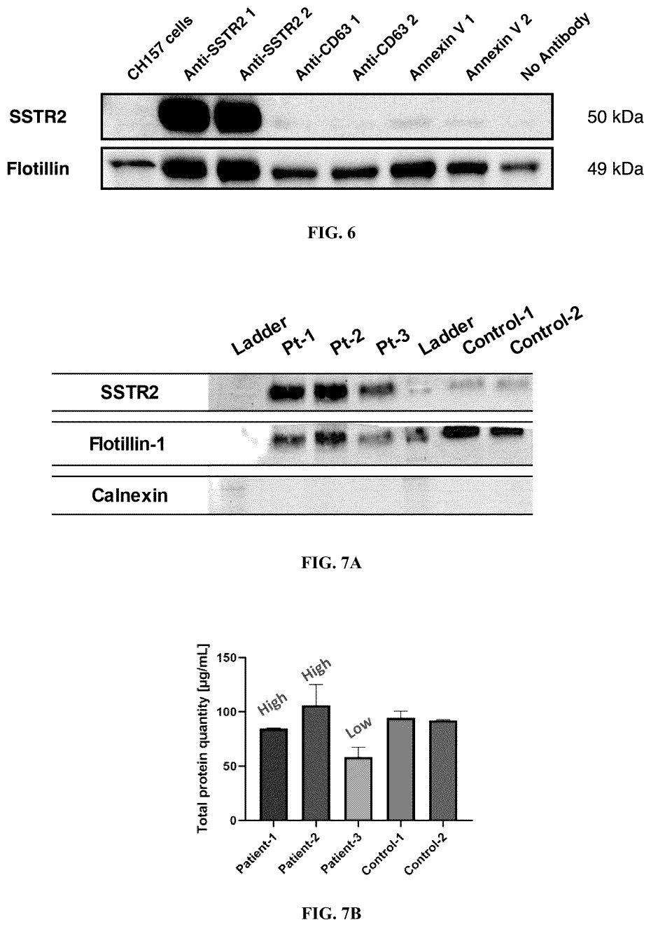

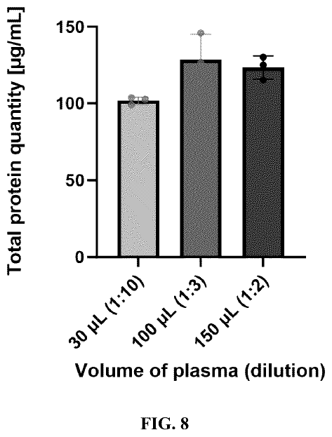

^{MGM}ExoChip was optimized for flow and capture conditions using EVs harvested from model MGM cells-CH157 and two primary cells (FIGS. 3 and 4). The capture efficiency of CH157 EVs was high (80.5±0.9%) compared to A549, lung cancer EVs (6.2±4.2%) using SSTR2 (FIG. 5). Among 3 antibodies anti-CD63, anti-SSTR2, and annexin-V, SSTR2 had the best capture of MGM EVs as confirmed using western blot (FIG. 6). Similarly, western blot confirmed that only vesicles from patient plasma samples expressing SSTR2 were captured and isolated using ^{MGM}ExoChip. 100 μL of plasma was sufficient to extract an average of 7 μg of proteins and 5 ng of RNA without saturating the device (FIG. 8 and Table 2). Western blot quantitation for 19 plasma samples (14 patients of high- and low-grade MGM, 5 healthy) and proteomics of 10 samples (7 patient, 3 healthy) was used to stratify MGM grades. mRNA sequencing of 6 samples showed further utility for risk stratification using molecular signatures.

TABLE 2
Concentration of RNA yields from
100 μL of plasma using ^{MGM}ExoChip.

| | | | DV200 (percentage of |
|---|---|---|---|
| Patient | Concentration | RNA integrity | RNA fragments >200 |
| ID | (ng/μL) | number (RIN) | nucleotides) |
| NU03629 | 0.24 | 2.5 | 24.0 |
| NU03780 | 0.16 | 1.2 | 30.0 |
| NU03643 | 0.31 | 2.4 | 48.0 |
| NU01391 | 0.15 | 1.2 | 60.0 |
| NU02242 | 0.13 | 2.3 | 3.0 |
| NU03639 | 0.16 | 2.4 | 3.0 |
| NU03607 | 0.22 | 2.6 | 37.0 |
| NU01563 | 0.15 | 2.5 | 9.0 |
| NU03635 | 0.2 | 2.5 | 22.0 |
| NU03293 | 0.05 | 2.8 | 36.0 |

### Example 3

Capture Experiments with Additional Molecules

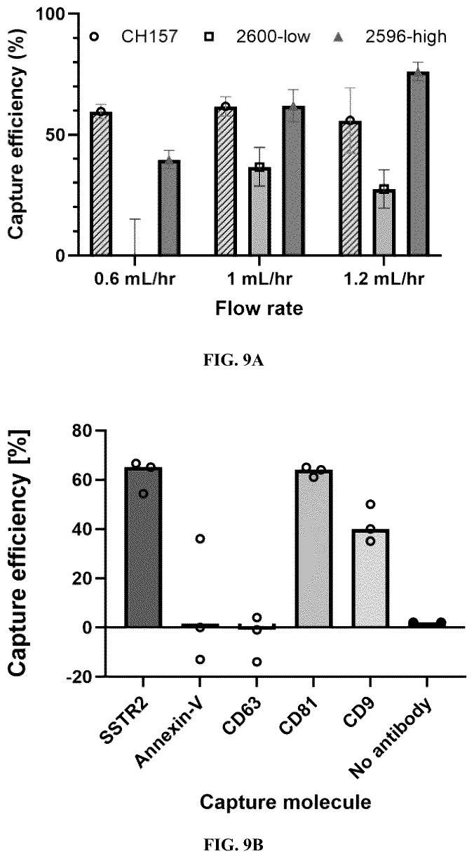

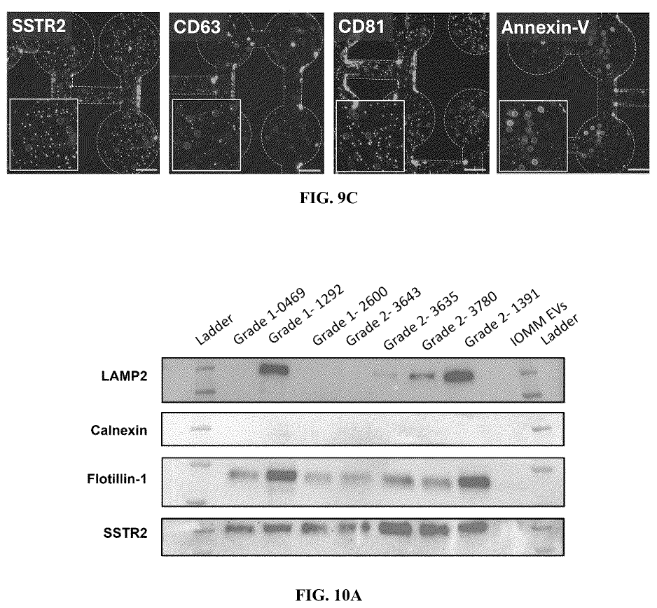

During the development of embodiments of the technology described herein, experiments were conducted to test capture of extracellular vesicles using different flow rates and different molecules. First, 3 flow rates were tested using EVs from 3 cell lines-1) CH157: immortalized meningioma cell line; 2) 2600: primary cells from grade 1 meningioma; and 3) 2596: primary cells from grade 2 meningioma. A flow rate of 1 mL/hr provided the most desirable capture efficiency for both grades of meningioma EVs. Nanoparticle tracking analysis (NTA) was used to obtain EV concentrations. FIG. 9A.

Then, capture efficiency of meningioma EVs from IOMM (immortalized meningioma cell line) was tested using three different capture molecules: CD63, CD81, and CD9, CD63, CD81, and CD9 are tetraspanins present on all EVs. As shown by FIG. 9B, CD81 provided the highest capture of these three test molecules, and the capture efficiency was comparable to the capture efficiency provided by SSTR2. Annexin-V, a molecule against phosphatidylserine that is reported to be overexpressed in cancer did not provide a significant or useful capture efficiency for meningioma EVs. FIG. 9B. Nanoparticle tracking analysis (NTA) was used to obtain EV concentrations. The data shown in FIG. 9C confirm the trends seen in FIG. 9B. Results were obtained by staining the EV membranes after they were captured on ^{MGM}ExoChip and imaged using a light microscope. The wells are marked using dotted outlines.

### Example 4

Western Blot Experiments with Grades 1 and 2 Meningioma EVs

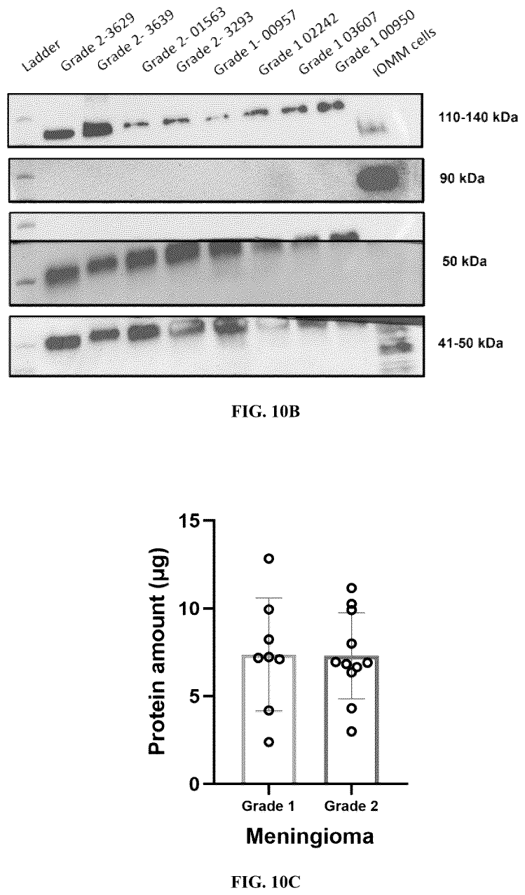

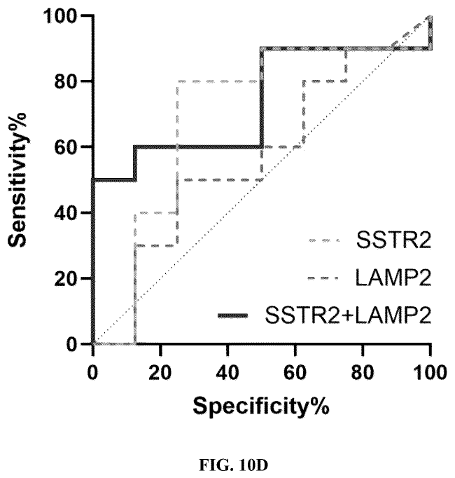

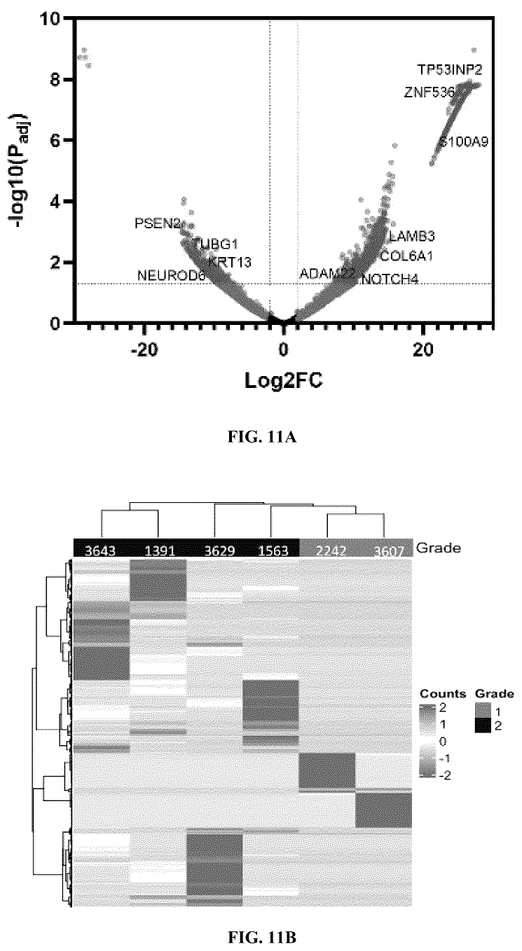

During the development of embodiments of the technology provided herein, experiments were conducted using 100 μL of plasma with SSTR2 as the capture molecule on a ^{MGM}ExoChip using a flow rate of 1 mL/hr. Protein was extracted using 100 μL RIPA buffer, protein quantification was performed using BCA assay, and equal amounts of proteins were loaded for western blot. FIG. 10A and FIG. 10B show that two proteins, SSTR2 and LAMP2, were expressed at different levels for the two grades of meningiomas.

Although the total protein amounts were similar (not significantly different) for the two grades (FIG. 10C), AUC-ROC analysis revealed that SSTR2 alone could distinguish between grade 1 and 2 meningiomas with 70% specificity (FIG. 10D). Adding LAMP2 increased the specificity to 74%.

### Example 5

mRNA Sequencing and Proteomics from Meningioma Plasma EVs

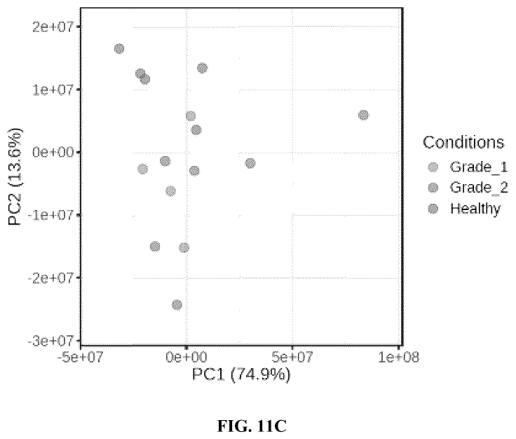

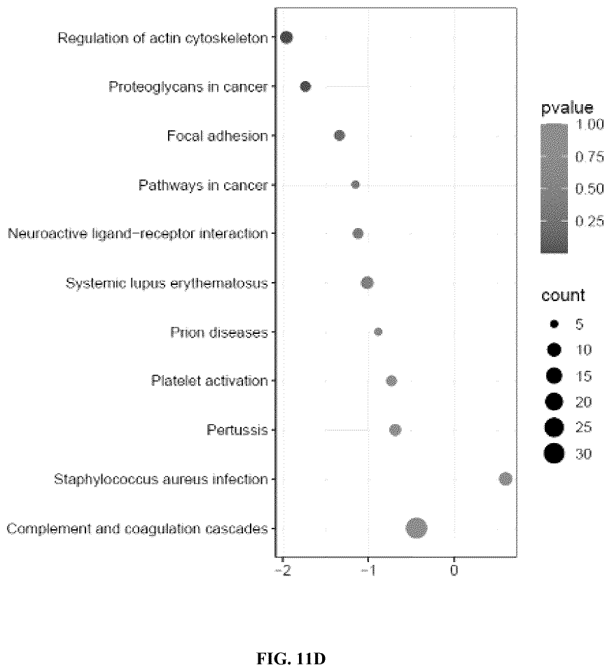

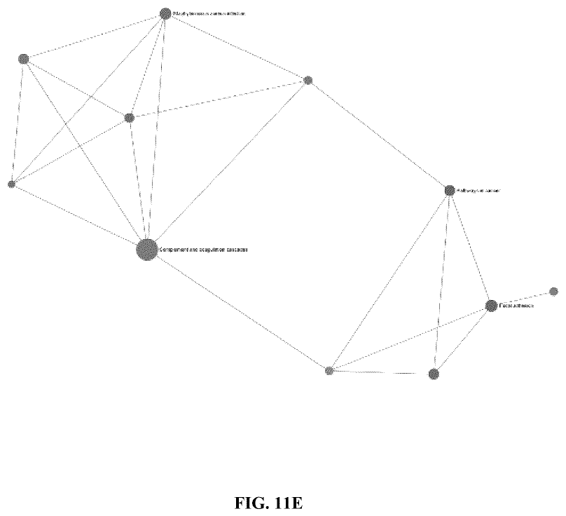

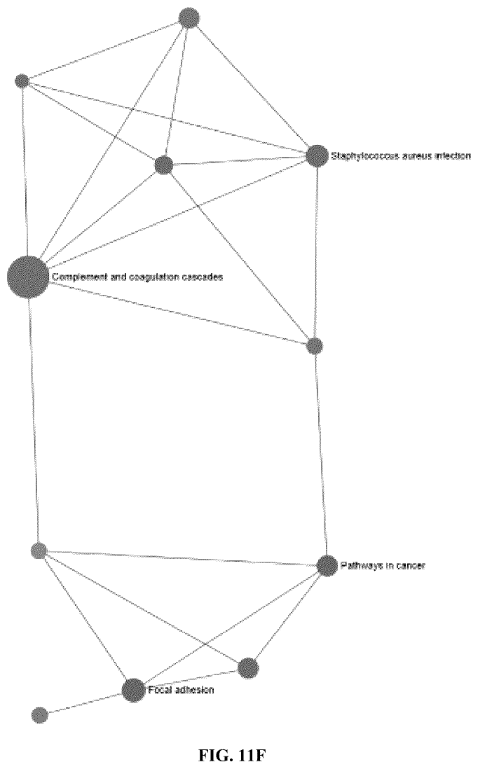

During the development of embodiments of the technology described herein, experiments were conducted using 100-μL plasma samples with SSTR2 as the capture molecule on ^{MGM}ExoChip at a flow rate of 1 mL/hr. Captured EVs were lysed on-chip using lysis buffer from Norgen Biotek's Exosomal RNA Isolation Kit. Six samples, comprising two grade 1 samples and four grade 2 samples, were sent to Norgen for next-generation sequencing (NGS). Differential expression analysis was then conducted to compare the grade 1 and grade 2 samples. FIG. 11A shows a volcano plot of differentially expressed genes. Cancer associated genes like ADAM22 and NOTCH4 are significantly upregulated in grade 2 meningioma EVs. FIG. 11B is a heatmap of differentially expressed genes presented after unsupervised clustering.

Next, similar experiments were conducted to prepare samples for proteomics. 75 μL of RIPA buffer was passed through captured EVs from four grade 1 meningioma samples, six grade 2 meningioma samples, and five healthy plasma samples. FIG. 11C shows that proteins from healthy EVs cluster together or with grade 1 meningioma. Gene set enrichment analysis for differentially expressed genes reveals genes involved in cancer, metastasis, and progression (FIG. 11D shows grade 1 versus grade 2; FIG. 11E shows grade 2 versus healthy; and FIG. 11F shows grade 1 versus healthy). Genes like APOD and APOE are highly expressed in both grades of meningiomas, and they have been reported to have prognostic values for this disease. Further, proteins corresponding to 3 genes that are significantly upregulated—H1-5, A2M, and FN1; and 3 proteins corresponding to three significantly downregulated genes—APOD, LBP, APOA4 in grade 2 compared to grade 1 are investigated further for their specificity in distinguishing between meningioma grades.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A microfluidic device for isolating extracellular vesicles comprising:
an inlet;
an outlet; and
a plurality of wells distributed between and fluidly connected to the inlet and outlet,
wherein each of the plurality of wells in fluid communication with two or more adjacent wells downstream of the inlet by a connecting channel, and
wherein the plurality of wells, connecting channels, or a combination thereof are functionalized with an extracellular vesicle capture agent.

2. The microfluidic device of claim 1, wherein the inlet is fluidly connected to a collection of inlet channels, wherein each inlet channel is directed to a different well proximal to the inlet.

3. The microfluidic device of claim 2, wherein the plurality of wells is configured to uniformly distribute sample flow from the inlet channels to the plurality of wells.

4. The microfluidic device of claim 1, wherein the plurality of wells is at least 2000 wells.

5. (canceled)

6. The microfluidic device of claim 1, wherein the plurality of wells has a volume capacity of greater than about 30 μL.

7. (canceled)

8. The microfluidic device of claim 1, wherein each of the plurality of wells has a volume capacity of about 4 nL to about 6 nL.

9. The microfluidic device of claim 1, wherein each of the plurality of wells has a diameter of about 200 μm to 400 μm.

10. The microfluidic device of claim 1, wherein the microfluidic device has a capacity for up to about 100 μL of plasma.

11. (canceled)

12. The microfluidic device of claim 1, wherein the extracellular vesicle capture agent is a binding partner of an extracellular vesicle marker, a cell marker, or a disease marker.

13. The microfluidic device of claim 1, wherein the extracellular vesicle capture agent is a binding partner of one or more membrane proteins.

14. The microfluidic device of claim 1, wherein the extracellular vesicle capture agent is an antibody, or fragment thereof.

15. A system comprising:
a device of claim 1; and
a fluid flow system.

16. A method for isolating extracellular vesicles of interest comprising:
loading a sample comprising or suspected of comprising the extracellular vesicles of interest into a device of claim 1; and
recovering the extracellular vesicles of interest, or components thereof, from the device.

17. The method of claim 16, wherein the sample is loaded at a flow rate of about 0.2 mL/hr to about 2.5 mL/hr.

18. (canceled)

19. The method of claim 16, wherein the sample has a residence time in the device of about 30 seconds to about 5 minutes.

20. (canceled)

21. The method of claim 16, further comprising analyzing molecular signatures of the recovered extracellular vesicles of interest, or components thereof.

22. The method of claim 21, wherein analyzing molecular signatures comprises proteomic analysis of proteins or nucleic acid sequencing.

23.-24. (canceled)

25. The method of claim 21, wherein analyzing molecular signatures comprises detecting a disease biomarker

26. The method of claim 16, wherein the sample is a biological sample.

27-30. (canceled)

31. The method of claim 16, wherein the extracellular vesicles of interest are cancer-derived extracellular vesicles.

32-48. (canceled)
